(19) United States
(12) Patent Application Publication
(10) Pub. No.: US 2026/0001944 A1
(43) Pub. Date: Jan. 1, 2026

(54) METHODS OF TREATING PAIN CAUSED BY A BONE FRACTURE

(71) Applicants: THE TRUSTEES OF INDIANA UNIVERSITY, Bloomington, IN (US); The United States of America as represented by the Department of Veterans Affairs Office of General, Washington, DC (US)

(72) Inventors: Fletcher Alan White, Indianapolis, IN (US); Jill Christine Fehrenbacher, Indianapolis, IN (US); Melissa Ann Kacena, Zionsville, IN (US); Roman M. Natoli, Indianapolis, IN (US); Kelly Marie Naugle, Indianapolis, IN (US)

(21) Appl. No.: 19/255,638

(22) Filed: Jun. 30, 2025

Related U.S. Application Data
(60) Provisional application No. 63/666,414, filed on Jul. 1, 2024.

(51) Int. Cl. C07K 16/18; A61K 39/00

(52) U.S. Cl. CPC C07K 16/18; A61K 2039/505; C07K 2317/24

(57) ABSTRACT

The inventors discovered that calcitonin gene-related peptide (CGRP) inhibitors reduce pain caused by bone fracture without impeding healing of the fracture. Accordingly, disclosed herein are methods for treating pain in a subject that has sustained a bone fracture, and the methods comprise administering a therapeutically effective amount of a CGRP inhibitor to the subject.

### CROSS-REFERENCE TO RELATED APPLICATIONS

The present application claims priority to U.S. Provisional Patent Application No. 63/666,414 that was filed Jul. 1, 2024, the entire contents of which are hereby incorporated by reference.

### STATEMENT REGARDING FEDERALLY SPONSORED RESEARCH OR DEVELOPMENT

Not applicable.

### BACKGROUND

Approaches to pain management following orthopedic surgery often include the use of opioids. The use of non-narcotic drugs such as nonsteroidal anti-inflammatory drugs (NSAIDs) for fracture pain is of limited use because of data suggesting that NSAIDs inhibit early fracture healing. Therefore, there is a need in the art for new analgesics that do not compromise bone healing or contribute to chronic pain conditions.

### SUMMARY

In an aspect of the current disclosure, methods are provided. In some embodiments, the methods comprise administering a therapeutically effective amount of a calcitonin gene-related peptide (CGRP) inhibitor to a subject that has sustained a bone fracture. In some embodiments, the CGRP inhibitor is a monoclonal antibody. In some embodiments, the CGRP inhibitor is selected from the group consisting of fremanezumab, erenumab, galcanezumab, eptinezumab, rimegepant, ubrogepant, atogepant, and zavegepant. In some embodiments, the CGRP inhibitor is selected from the group consisting of fremanezumab, erenumab, galcanezumab, and eptinezumab. In some embodiments, the CGRP inhibitor is fremanezumab. In some embodiments, the method does not negatively impact the healing of the bone fracture. In some embodiments, the subject sustained the bone fracture about 6 months to about 1 hour prior to administration of the CGRP inhibitor.

In an aspect of the current disclosure, methods of treating pain in a subject that has experienced a bone fracture are provided. In some embodiments, the methods comprise administering a therapeutically effective amount of a calcitonin gene-related peptide (CGRP) inhibitor to the subject to treat pain in the subject. In some embodiments, the CGRP inhibitor is a monoclonal antibody. In some embodiments, the CGRP inhibitor is selected from the group consisting of fremanezumab, erenumab, galcanezumab, eptinezumab, rimegepant, ubrogepant, atogepant, and zavegepant. In some embodiments, the CGRP inhibitor is selected from the group consisting of fremanezumab, erenumab, galcanezumab, and eptinezumab. In some embodiments, the CGRP inhibitor is fremanezumab. In some embodiments, the method does not negatively impact the healing of the bone fracture.

In an aspect of the current disclosure, methods of treating pain in a subject with a bone fracture without inhibiting healing of the fracture are provided. In some embodiments, the methods comprise administering a therapeutically effective amount of a calcitonin gene-related peptide (CGRP) inhibitor to the subject to treat pain in the subject without inhibiting healing of the fracture. In some embodiments, the CGRP inhibitor is a monoclonal antibody. In some embodiments, the CGRP inhibitor is selected from the group consisting of fremanezumab, erenumab, galcanezumab, eptinezumab, rimegepant, ubrogepant, atogepant, and zavegepant. In some embodiments, the CGRP inhibitor is selected from the group consisting of fremanezumab, erenumab, galcanezumab, and eptinezumab. In some embodiments, the CGRP inhibitor is fremanezumab. In some embodiments, the subject experienced a bone fracture about 1 week to about 1 hour before administration of the CGRP inhibitor. In some embodiments, the subject experienced a bone fracture about 1 day to about 1 hour before administration of the CGRP inhibitor.

In an aspect of the current disclosure, methods of preventatively treating pain in a subject that will experience a bone fracture are provided. In some embodiments, the methods comprise administering a therapeutically effective amount of a calcitonin gene-related peptide (CGRP) inhibitor to the subject to preventatively treat pain in the subject. In some embodiments, the CGRP inhibitor is a monoclonal antibody. In some embodiments, the CGRP inhibitor is selected from the group consisting of fremanezumab, erenumab, galcanezumab, eptinezumab, rimegepant, ubrogepant, atogepant, and zavegepant. In some embodiments, the CGRP inhibitor is selected from the group consisting of fremanezumab, erenumab, galcanezumab, and eptinezumab. In some embodiments, the CGRP inhibitor is fremanezumab. In some embodiments, the method does not negatively impact the healing of the bone fracture. In some embodiments, the subject is receiving an orthopedic surgery and the CGRP inhibitor is administered at about the time of the orthopedic surgery.

In an aspect of the current disclosure, methods of treating pain in a subject that has undergone or will undergo orthopedic surgery are provided. In some embodiment, the methods comprise administering a therapeutically effective amount of a calcitonin gene-related peptide (CGRP) inhibitor to the subject to treat pain in the subject. In some embodiments, the CGRP inhibitor is a monoclonal antibody. In some embodiments, the CGRP inhibitor is selected from the group consisting of fremanezumab, erenumab, galcanezumab, eptinezumab, rimegepant, ubrogepant, atogepant, and zavegepant. In some embodiments, the CGRP inhibitor is selected from the group consisting of fremanezumab, erenumab, galcanezumab, and eptinezumab. In some embodiments, the CGRP inhibitor is fremanezumab. In some embodiments, the method does not negatively impact the healing of the bone fracture. In some embodiments, the method reduces chronic pain due to the bone fracture in the subject. In some embodiments, the CGRP inhibitor is administered at about the time of surgery to about 6 months after the surgery.

### BRIEF DESCRIPTION OF THE FIGURES

FIGS. 1A, 1B, and 1C show that fremanezumab does not compromise rate of bone union but does attenuate fracture-induced spontaneous pain behaviors. A) Mouse grimace scores were assessed after surgically-created fracture of the right femur in mice treated with buprenorphine SR or fremanezumab. B) Dynamic weightbearing was assessed and is reported as % weightbearing on the injured hindlimb. C) Bone union was assessed by mRUST scoring of femoral x-rays. Mean #SEM, P<0.05 for effects of surgery, as determined by two-way ANOVA and Dunnett's post-hoc test for multiple comparisons at each timepoint, **** P<0.0001, ** P<0.01 and *P<0.05, n=8-10 for each treatment condition.

FIG. 2 shows that CGRP levels show a trending increase in patient plasma following fracture. Plasma was collected 2 weeks following surgery from patients with femur or tibia fractures treated with intramedullary nail (orange dots) or control patients (green dots) and assessed for CGRP via ELISA. n=10-21 per group.

FIG. 3 shows Plasma levels of CGRP measured 2 weeks after lower extremity long bone fracture in fracture patients without chronic pain, fracture patients reporting ongoing pain >6 months post-injury, and healthy controls.

### DETAILED DESCRIPTION

Treating pain resulting from a bone fracture with non-steroidal anti-inflammatory drugs (NSAIDS), while effective as an analgesic, inhibits the early healing of the fractured bone. Furthermore, bone fractures, even when completely healed, can result in chronic pain in the subject.

The neuropeptide named calcitonin gene-related peptide (CGRP) is expressed in central and peripheral nervous tissue. Several CGRP inhibitors have been approved for treating migraine headaches. The inventors discovered that calcitonin gene-related peptide (CGRP) inhibitors are effective at treating pain caused by surgical fracture of the femur, using a mouse model of bone fracture pain.

Accordingly, disclosed herein are methods of treating pain in a subject that has experienced a bone fracture, methods of inducing analgesia in a subject with a bone fracture without inhibiting healing of the fracture, methods of preventatively treating pain in a subject that will experience a bone fracture, and method of treating pain in a subject that has undergone or will undergo orthopedic surgery.

### Methods

In an aspect of the current disclosure, methods are provided. In some embodiments, the methods comprise administering a therapeutically effective amount of a calcitonin gene-related peptide (CGRP) inhibitor to a subject that has sustained a bone fracture.

As used herein, a “calcitonin gene-related peptide (CGRP) inhibitor” refers to a composition that interferes with the biological action of CGRP. For example, some CGRP inhibitors bind to CGRP itself and prevent ligation of CGRP to its receptor: calcitonin receptor-like receptor (CALCRL) or receptor activity-modifying protein (RAMP1). Another group of CGRP inhibitors bind to a receptor of CGRP and prevent ligation of CGRP to its receptor. Several CGRP inhibitors have been approved for use in humans, e.g., fremanezumab, erenumab, galcanezumab, eptinezumab, rimegepant, ubrogepant, atogepant, and zavegepant. Fremanezumab, erenumab, galcanezumab, and eptinezumab are monoclonal antibodies that bind directly to CGRP and inhibit binding of CGRP to its receptor. Rimegepant, ubrogepant, atogepant, and zavegepant are small molecules that inhibit binding of CGRP to its receptor.

As used herein, a “therapeutically effective amount” or an “effective amount” refers to the amount of the CGRP inhibitor necessary to improve one or more sign or symptom of a particular disease or disorder, e.g., pain caused by a bone fracture. A therapeutically effective amount may be determined by a prescribing physician empirically within doses that have been demonstrated to be safe, which is within the ordinary skill in the art. The CGRP inhibitors may be administered by any appropriate route, e.g., parenterally, e.g., subcutaneously. Illustrative routes of administration include transdermal, percutaneous, intravenous, intramuscular, intranasal, buccal, intrathecal, intracerebral, oral, or intrarectal routes. The administration route can be determined by a physician.

TABLE 1
Exemplary human dosages for CGRP inhibitors. The following
doses are based on the U.S. Food and Drug Administration
(FDA) approved treatment guidelines but may be modified
at the discretion of a physician.

| CGRP inhibitor | Exemplary human dose |
|---|---|
| fremanezumab | About 100 mg to about 300 mg, once per |
| | month. About 300 mg to about 900 mg |
| | administered once every three months. 225 |
| | mg may be administered once per month |
| | subcutaneously. |
| | The inventors demonstrated that 30 mg/kg |
| | fremanezumab administered daily was |
| | effective at reducing pain associated with a |
| | fracture in a mouse model, which is |
| | equivalent to about 2.44 mg/kg |
| | administered to a human, using a standard |
| | conversion to a human dose based on body |
| | surface area (mouse dose/12.3 = human |
| | dose; see Nair and Jacob “A simple practice |
| | guide for dose conversion between animals |
| | and human” J Basic Clin Pharm. 2016 |
| | March; 7(2): 27-31, which is incorporated by |
| | reference herein in its entirety. For a |
| | standard 60 kg human, this equates to about |
| | 146 mg fremanezumab. |
| erenumab | About 50 mg to about 200 mg. May be |
| | administered once monthly. May be about |
| | 70 mg to about 140 mg. May be 70 mg. |
| galcanezumab, | About 100 to about 300 mg. About 120 mg |
| | to about 240 mg. About 240 mg or about |
| | 120 mg. May be given as about 240 mg |
| | loading dose. May be administered once |
| | monthly. |
| eptinezumab | About 50 mg to about 500 mg. About 100 |
| | to about 300 mg. |
| rimegepant | About 25 to about 125 mg or about 75 mg. |
| ubrogepant | About 25 to about 125 mg or about 50 to |
| | about 100 mg. About 50 mg as an initial |
| | dose and another 50 mg taken as needed for |
| | a second dose. About 100 mg as an initial |
| | dose and another 50 mg taken as needed for |
| | a second dose. |
| atogepant | About 5 to about 100 mg. About 10, about |
| | 30, or about 60 mg, may be taken once |
| | daily. |
| zavegepant | About 1 to about 100 mg or about 10 mg. |
| | May be formulated for intranasal |
| | administration. |

As used herein, a “subject” or a “subject in need thereof” may refer to a subject that has experienced a bone fracture or a subject that is expected to experience a bone fracture, e.g., a subject that is scheduled to receive orthopedic surgery or a subject that has received orthopedic surgery where a bone is cut or otherwise damaged in a way that could lead to pain. A bone fracture may comprise any type of fracture, e.g., open fractures, closed fractures, stress fractures. The subject may comprise a subject with a bone fracture that is treated with a cast, splint, or other non-surgical intervention. The subject may be a human subject. The subject may be non-human animal, e.g., a mammal, e.g., a horse, a dog, a cat, a goat, a donkey, etc.

The disclosed methods may be performed on a subject that has experienced a bone fracture or orthopedic surgery recently, e.g., about 6 months, about 5 months, about 4 months, about 3 months, about 2 months, about 1 month, about 4 weeks, about 3 weeks, about 2 weeks, about 1 week, about 6 days, about 5 days, about 4 days, about 3 days, about 2 days about 1 day, or less before administration of the CGRP inhibitor. The disclosed methods may be performed on a subject that has experienced a bone fracture or orthopedic surgery recently, e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours prior to administration of the CGRP inhibitor.

In some embodiments, the methods are methods of treating pain in a subject that has experienced a bone fracture and the methods comprise administering a therapeutically effective amount of a calcitonin gene-related peptide (CGRP) inhibitor to the subject to treat pain in the subject.

Methods of treating pain in a subject with a bone fracture without inhibiting healing of the fracture are provided and comprise administering a therapeutically effective amount of a calcitonin gene-related peptide (CGRP) inhibitor to the subject to treat pain in the subject without inhibiting healing of the fracture.

As used herein, “treating pain,” and grammatical variations thereof, refers to a reduction in perceived pain by the subject or a reduction in signs associated with pain in the subject. In some embodiments, treating pain refers to treating pain associated with a bone fracture. In some embodiments, the disclosed methods treat “chronic pain” from a bone fracture which is differentiated from the pain resulting immediately from the bone fracture (acute, described above) and can be experienced by the subject following healing of the bone fracture. Chronic pain may occur in the subject about 2 weeks, about 1 month, about 2 months, about 3 months, about 4 months, about 5 months, about 6 months, about 7 months, about 8 months, about 9 months, about 10 months, about 11 months, about 12 months, or more following a bone fracture or following healing of a bone fracture.

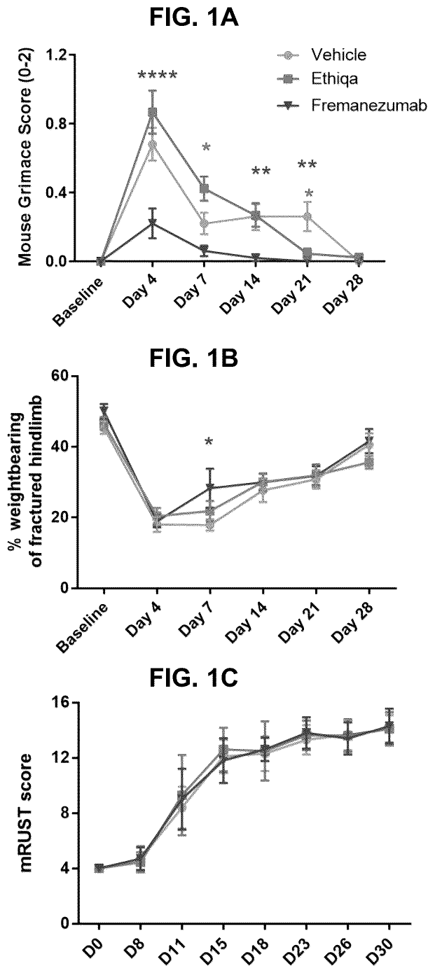

As used herein, “inducing analgesia without inhibiting healing of the fracture” refers to reducing the sensation of pain in the subject due to the fracture while not negatively impacting the normal healing processes of an early bone fracture, in contrast to NSAIDs which, as discussed above, have been shown to negatively impact the early healing processes of a bone fracture. The inventors demonstrated that the CGRP inhibitor fremanezumab reduces signs of pain due to fracture of the femur (FIG. 1A) but does not impact healing of the fracture, i.e., the time to bone union (FIG. 1C) as compared to the opioid buprenorphine (noted as ETHIQA in FIG. 1, which is an injectable sustained-release form of buprenorphine) and as compared to vehicle control. Put another way, administration of the CGRP inhibitor does not negatively impact healing of the bone fracture in the subject as compared to a subject with the same or similar fracture and that has not been administered CGRP inhibitor.

In some embodiments, the methods are methods of treating pain in a subject that has undergone or will undergo orthopedic surgery and the methods comprise administering a therapeutically effective amount of a calcitonin gene-related peptide (CGRP) inhibitor to the subject to treat pain in the subject.

As used herein, a “subject that has undergone or will undergo orthopedic surgery” refers to a subject that has undergone orthopedic surgery, e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours prior to administration of the CGRP inhibitor. In some embodiments, the subject has undergone orthopedic surgery about 1 day, about 2 days, about 3 days, about 4 days, about 5 days, about 6 days, about 7 days or more prior to administration of the CGRP inhibitor. In some embodiments, the subject has undergone orthopedic surgery about 1 week, about 2 weeks, about 3 weeks, about 4 weeks, or more prior to administration of the CGRP inhibitor. In some embodiments, the subject has undergone orthopedic surgery about 1 month, about 2 months, about 3 months, about 4 months, about 5 months, about 6 months, or more prior to administration of the CGRP inhibitor. In some embodiments, the subject has undergone orthopedic surgery about 4 weeks, about 3 weeks, about 2 weeks about 1 week, about 6 days, about 5 days, about 4 days, about 3 days, about 2 days, or about 1 day prior to administration of the CGRP inhibitor.

In some embodiments, the disclosed methods are used to prevent pain in the subject by administering the CGRP inhibitor before the surgery or at about the time of surgery, e.g., as prophylaxis for pain caused by the surgery. For example, the CGRP inhibitor may be administered immediately preceding the orthopedic surgery, during the orthopedic surgery, e.g., while the subject is anesthetized, or immediately following the orthopedic surgery. See also FIG. 1 and Example 1 where the inventors administered fremanezumab at the time of surgical femur fracture resulting in reduced pain in the animal subjects.

The administration of the CGRP inhibitor may be continued as needed to treat pain in the subject.

The surgical treatment may include, for example, hip replacement surgery, knee replacement surgery, anterior cruciate ligament (ACL) reconstruction, shoulder replacement surgery, knee arthroscopy, shoulder arthroscopy, ankle repair, spinal surgery, joint fusion, or trigger finger release.

The disclosed methods may further comprise administering another drug in combination with the CGRP inhibitor, e.g., an opioid, an antibiotic, an antihistamine, acetaminophen, or other suitable drug. NSAIDs may be administered in combination with the CGRP inhibitor, but may negatively impact bone fracture healing. The CGRP inhibitors may be administered with gabapentin, pregabalin, or with an antidepressant or antipsychotic. The CGRP inhibitor may be co-administered with an opioid or an NSAID. The half-life of the CGRP inhibitor may limit how often the CGRP inhibitor could be administered. Other pain relievers, e.g., opioids, could be used as a ‘rescue’ in cases of inadequate pain control.

The disclosed methods of administering a CGRP inhibitor may also be used for the treatment of pain associated with dental procedures, with ear nose and throat (ENT) related procedures, or treatment of pain for any injury/procedure.

### Further Definitions

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

### ILLUSTRATIVE EMBODIMENTS

1. A method comprising administering a therapeutically effective amount of a calcitonin gene-related peptide (CGRP) inhibitor to a subject that has sustained a bone fracture.
2 The method of embodiment 1, wherein the CGRP inhibitor is a monoclonal antibody.
3. The method of embodiment 1, wherein the CGRP inhibitor is selected from the group consisting of fremanezumab, erenumab, galcanezumab, eptinezumab, rimegepant, ubrogepant, atogepant, and zavegepant.
4. The method of embodiment 3, wherein the CGRP inhibitor is selected from the group consisting of fremanezumab, erenumab, galcanezumab, and eptinezumab.
5. The method of embodiment 4, wherein the CGRP inhibitor is fremanezumab.
6. The method of any one of embodiments 1-5, wherein the method does not negatively impact the healing of the bone fracture.
7. The method of any one of embodiments 1-6, wherein the subject sustained the bone fracture about 6 months to about 1 hour prior to administration of the CGRP inhibitor.
8. A method of treating pain in a subject that has experienced a bone fracture, the method comprising administering a therapeutically effective amount of a calcitonin gene-related peptide (CGRP) inhibitor to the subject to treat pain in the subject.
9. The method of embodiment 8, wherein the CGRP inhibitor is a monoclonal antibody.
10. The method of embodiment 8, wherein the CGRP inhibitor is selected from the group consisting of fremanezumab, erenumab, galcanezumab, eptinezumab, rimegepant, ubrogepant, atogepant, and zavegepant.
11. The method of embodiment 10, wherein the CGRP inhibitor is selected from the group consisting of fremanezumab, erenumab, galcanezumab, and eptinezumab.
12. The method of embodiment 11, wherein the CGRP inhibitor is fremanezumab.
13. The method of any one of embodiments 8-12, wherein the method does not negatively impact the healing of the bone fracture.
14. A method of treating pain in a subject with a bone fracture without inhibiting healing of the fracture, the method comprising administering a therapeutically effective amount of a calcitonin gene-related peptide (CGRP) inhibitor to the subject to treat pain in the subject without inhibiting healing of the fracture.
15. The method of embodiment 14, wherein the CGRP inhibitor is a monoclonal antibody.
16. The method of embodiment 14, wherein the CGRP inhibitor is selected from the group consisting of fremanezumab, erenumab, galcanezumab, eptinezumab, rimegepant, ubrogepant, atogepant, and zavegepant.
17. The method of embodiment 16, wherein the CGRP inhibitor is selected from the group consisting of fremanezumab, erenumab, galcanezumab, and eptinezumab.
18. The method of embodiment 17, wherein the CGRP inhibitor is fremanezumab.
19. The method of any one of embodiments 14-18, wherein the subject experienced a bone fracture about 1 week to about 1 hour before administration of the CGRP inhibitor.
20. The method of embodiment 19, wherein the subject experienced a bone fracture about 1 day to about 1 hour before administration of the CGRP inhibitor.
21. A method of preventatively treating pain in a subject that will experience a bone fracture, the method comprising administering a therapeutically effective amount of a calcitonin gene-related peptide (CGRP) inhibitor to the subject to preventatively treat pain in the subject.
22. The method of embodiment 21, wherein the CGRP inhibitor is a monoclonal antibody.
23. The method of embodiment 21, wherein the CGRP inhibitor is selected from the group consisting of fremanezumab, erenumab, galcanezumab, eptinezumab, rimegepant, ubrogepant, atogepant, and zavegepant.
24. The method of embodiment 23, wherein the CGRP inhibitor is selected from the group consisting of fremanezumab, erenumab, galcanezumab, and eptinezumab.
25. The method of embodiment 24, wherein the CGRP inhibitor is fremanezumab.
26. The method of any one of embodiments 21-25, wherein the method does not negatively impact the healing of the bone fracture.
27. The method of any one of embodiments 21-26, wherein the subject is receiving an orthopedic surgery and the CGRP inhibitor is administered at about the time of the orthopedic surgery.
28. A method of treating pain in a subject that has undergone or will undergo orthopedic surgery, the method comprising administering a therapeutically effective amount of a calcitonin gene-related peptide (CGRP) inhibitor to the subject to treat pain in the subject.
29. The method of embodiment 28, wherein the CGRP inhibitor is a monoclonal antibody.
30. The method of embodiment 28, wherein the CGRP inhibitor is selected from the group consisting of fremanezumab, erenumab, galcanezumab, eptinezumab, rimegepant, ubrogepant, atogepant, and zavegepant.
31. The method of embodiment 30, wherein the CGRP inhibitor is selected from the group consisting of fremanezumab, erenumab, galcanezumab, and eptinezumab.
32. The method of embodiment 31, wherein the CGRP inhibitor is fremanezumab.
33. The method of any one of embodiments 28-32, wherein the method does not negatively impact the healing of the bone fracture.
34. The method of any one of embodiments 28-33, wherein the method reduces chronic pain due to the bone fracture in the subject.
35. The method of any one of embodiments 28-34, wherein the CGRP inhibitor is administered at about the time of surgery to about 6 months after the surgery.

### EXAMPLES

The following Examples are illustrative and should not be interpreted to limit the scope of the claimed subject matter.

### Example 1—The CGRP Inhibitor Fremanezumab Attenuates Fracture-Induced Pain Behavior but does not Compromise Fracture Healing

Sequestering CGRP abates fracture-induced pain behaviors to a greater extent than the veterinary standard-of-care, buprenorphine SR, without gross deficits in fracture healing. To determine whether sequestering CGRP attenuates fracture pain behaviors, we administered fremanezumab, a monoclonal antibody against CGRP, recently approved by the FDA for migraine prophylaxis. This drug has a distinct advantage for our proposed study as the antibody scavenges free circulating CGRP in rodent species.⁵ In FIG. 1, we asked whether treatment of mice with veterinary standard-of-care treatments of the opioid, buprenorphine SR (ETHIQA XR; 3.25 mg/kg injected at surgery), or fremanezumab (30 mg/kg daily, subcutaneous (sc)) would alter the development of pain behaviors measured from day 4 to day 28. In FIG. 1A, spontaneous pain is assessed by facial grimace scoring, as described previously.^{1,2} There is a large increase in grimace score within the vehicle-treated group (green symbols and lines) at day 4 and a slow resolution of this indicator of sharp spontaneous pain towards day 28. The buprenorphine did not significantly affect grimace behavior in the mice; however, mice treated with fremanezumab had a profound reduction in grimace score that persisted through the course of fracture healing. Indeed, spontaneous pain in these animals, as assessed by grimace score, was significantly lower at day 4 and had almost returned to baseline by day 7 following fracture. In FIG. 1B, the baseline measurements prior to fracture demonstrate that weightbearing is equal between the left and the right hindlimbs with 50% of the weight borne on each hindlimb. Following fracture, there is an obvious preference for unloading the fractured hindlimb in vehicle-treated fracture animals. Fremanezumab has a small effect to enhance weightbearing on Day 7, but not at the later days.

We also determined that sequestering CGRP with fremanezumab did not impair bone healing in the mice. Longitudinal femoral x-rays were scored for fracture healing, as described previously.⁵ For this mRUST scoring, a score of 16 would indicate fracture healing with complete remodeling.⁵ Radiographic scoring indicates that neither buprenorphine nor fremanezumab altered the time course of fracture healing, as assessed by mRUST scoring (FIG. 1C).

Altogether, these data demonstrate that fremanezumab has potential to reduce the onset of fracture-associated pain behaviors without compromising the time to fracture union, as assessed via x-rays.

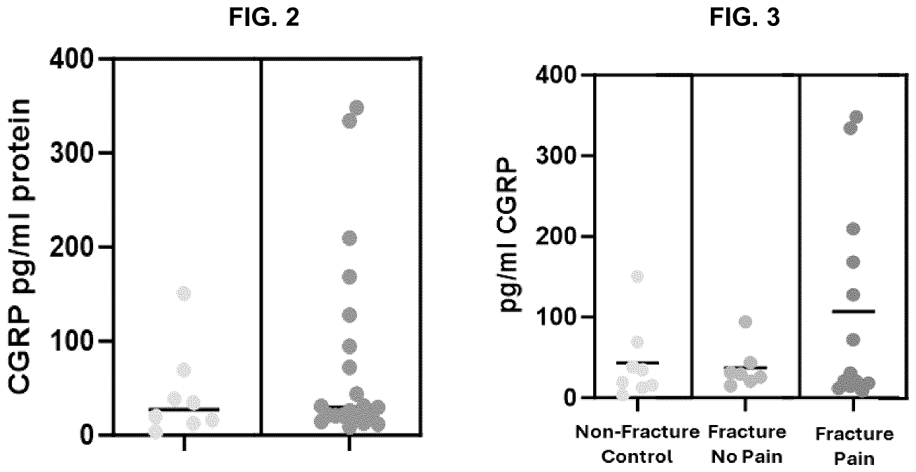

Trending elevation in CGRP 2 weeks after lower extremity long bone fractures. A fundamental premise of the use of anti-CGRP agents for fracture pain is that elevated levels of CGRP contribute to fracture pain. Therefore, we examined circulating levels of CGRP in a clinical fracture population compared with age and sex matched injury-free control subjects and then correlated early levels of these factors with the development of ongoing pain in the patients. Plasma was obtained from 20 patients aged 18-60 inclusive who sustained lower extremity long bone fractures (i.e., femur distal to the intertrochanteric region or tibia) treated with IMNs. All fracture patients had uncomplicated post-operative courses and were followed for >6 months until bone healing was complete. As illustrated in FIG. 2, patients with fracture trended towards increased circulating plasma concentrations of CGRP compared to plasma levels in non-fractured control patients.

Clearly, this is just a snapshot in time, and our ongoing studies will further evaluate the time course of CGRP changes in fracture patients over time.

In FIG. 2, we examined differences in circulating CGRP between fracture patients and healthy control patients ˜2 weeks following fracture surgery. All fracture patients had VAS (0-10) pain ratings collected >6 months after surgery. We stratified patients into fracture groups without pain (VAS=0 at 6 months, n=7) or with pain (VAS>0, n=14). In FIG. 3, we highlight the differences in CGRP levels between non-fractured subjects and fracture patients that went on to heal their fracture but were subdivided into groups that resolved their pain vs. those with ongoing fracture pain. We found trending elevations in CGRP in patients with chronic pain after fracture healing compared to those without pain (p=0.078), with a moderate effect size between groups (Cohen's d=0.63). Of note, no significant or trending differences were observed between non-fractured subjects and fractured patients with resolved pain. With a larger sample size, we expect differences in CGRP to emerge within 2 weeks of fracture between fracture patients who develop chronic pain compared to those who do not, with CGRP following different curves across time.

### REFERENCES

1. Langford D J, Bailey A L, Chanda M L, et al. Coding of facial expressions of pain in the laboratory mouse. Nat Methods. 2010; 7 (6): 447-449.
2. Nguyen T, Nguyen N, Cochran A G, et al. Repeated closed-head mild traumatic brain injury-induced inflammation is associated with nociceptive sensitization. J Neuroinflammation. 2023; 20 (1): 196.
3. Kasai Y, Aso K, Izumi M, et al. Increased Calcitonin Gene-Related Peptide Expression in DRG and Nerve Fibers Proliferation Caused by Nonunion Fracture in Rats. J Pain Res. 2021; 14:3565-3571.
4. Alentado V J, Knox A M, Staut C A, et al. Validation of the modified radiographic union score for tibia fractures (mRUST) in murine femoral fractures. Front Endocrinol (Lausanne). 2022; 13:911058.
5. Grell A S, Haanes K A, Johansson S E, Edvinsson L, Sams A. Fremanezumab inhibits vasodilatory effects of CGRP and capsaicin in rat cerebral artery-Potential role in conditions of severe vasoconstriction. Eur J Pharmacol. 2019; 864:172726.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

1. A method comprising administering a therapeutically effective amount of a calcitonin gene-related peptide (CGRP) inhibitor to a subject that has sustained a bone fracture.

2. The method of claim 1, wherein the CGRP inhibitor is a monoclonal antibody.

3. The method of claim 1, wherein the CGRP inhibitor is selected from the group consisting of fremanezumab, erenumab, galcanezumab, eptinezumab, rimegepant, ubrogepant, atogepant, and zavegepant.

4. The method of claim 3, wherein the CGRP inhibitor is selected from the group consisting of fremanezumab, erenumab, galcanezumab, and eptinezumab.

5. The method of claim 4, wherein the CGRP inhibitor is fremanezumab.

6. The method of claim 1, wherein the method does not negatively impact the healing of the bone fracture.

7. The method of claim 1, wherein the subject sustained the bone fracture about 6 months to about 1 hour prior to administration of the CGRP inhibitor.

8. A method of treating pain in a subject that has experienced a bone fracture, the method comprising administering a therapeutically effective amount of a calcitonin gene-related peptide (CGRP) inhibitor to the subject to treat pain in the subject.

9. The method of claim 8, wherein the CGRP inhibitor is a monoclonal antibody.

10. The method of claim 8, wherein the CGRP inhibitor is selected from the group consisting of fremanezumab, erenumab, galcanezumab, eptinezumab, rimegepant, ubrogepant, atogepant, and zavegepant.

11. The method of claim 10, wherein the CGRP inhibitor is selected from the group consisting of fremanezumab, erenumab, galcanezumab, and eptinezumab.

12. The method of claim 11, wherein the CGRP inhibitor is fremanezumab.

13. The method of claim 8, wherein the method does not negatively impact the healing of the bone fracture.

14. A method of treating pain in a subject with a bone fracture without inhibiting healing of the fracture, the method comprising administering a therapeutically effective amount of a calcitonin gene-related peptide (CGRP) inhibitor to the subject to treat pain in the subject without inhibiting healing of the fracture.

15. The method of claim 14, wherein the CGRP inhibitor is a monoclonal antibody.

16. The method of claim 14, wherein the CGRP inhibitor is selected from the group consisting of fremanezumab, erenumab, galcanezumab, eptinezumab, rimegepant, ubrogepant, atogepant, and zavegepant.

17. The method of claim 16, wherein the CGRP inhibitor is selected from the group consisting of fremanezumab, erenumab, galcanezumab, and eptinezumab.

18. The method of claim 17, wherein the CGRP inhibitor is fremanezumab.

19. The method of any one of claim 14, wherein the subject experienced a bone fracture about 1 week to about 1 hour before administration of the CGRP inhibitor.

20. The method of claim 19, wherein the subject experienced a bone fracture about 1 day to about 1 hour before administration of the CGRP inhibitor.
